(19) United States
(12) Patent Application Publication
(10) Pub. No.: US 2016/0061825 A1
(43) Pub. Date: Mar. 3, 2016

(54) Kits for Fecal Neopterin Concentration Measurement as an Indicator of Disease Activity in Inflammatory Bowel Disease

(71) Applicant: Northwestern University, Evanston, IL (US)

(72) Inventor: Alan L. Buchman, Northfield, IL (US)

(73) Assignee: NORTHWESTERN UNIVERSITY, Evanston, IL (US)

(21) Appl. No.: 14/935,655

(22) Filed: Nov. 9, 2015

Related U.S. Application Data
(63) Continuation of application No. 13/189,685, filed on Jul. 25, 2011, now abandoned.

(51) Int. Cl. G01N 33/53; G01N 33/49; G01N 15/05

(52) U.S. Cl. CPC G01N 33/5308; G01N 15/05; G01N 33/492; G01N 2800/065; G01N 2015/055; G01N 2333/805; G01N 2333/765; G01N 2333/4737; G01N 2333/4727; G01N 2333/79; G01N 2333/46

(57) ABSTRACT

Disclosed are methods for determining disease activity in a patient having or at risk for developing inflammatory bowel disease (IBD) which include measuring neopterin concentration in a fecal sample from the patient.

### BACKGROUND

The field of the invention relates to methods for determining disease activity in a patient having or at risk for developing inflammatory bowel disease. In particular, the field of the invention relates to methods for determining disease activity via fecal neopterin concentration measurement in patient having or at risk for developing Crohn's disease or ulcerative colitis.

Inflammatory bowel disease (IBD) is a group of inflammatory conditions of the colon and small intestine. The major types of IBD are Crohn's disease and ulcerative colitis. Both are chronic diseases that are characterized by intermittent periods of disease activity and remission. Relapses can be difficult to diagnose, especially by non-invasive testing and incorrect diagnosis may lead to over- or under-treatment. These diseases afflict an estimated 1-1.3 million Americans as well as others throughout the world.

A few small studies in the past have suggested urine neopterin may be an indicator of the presence of active disease. Less data is available concerning the use of serum neopterin as a measure of the presence of disease activity. Fecal neopterin has been used as an indicator of bacteria gastroenteritis in children of Gambia. However, bacteria gastroenteritis is unrelated to IBD.

Here, measurement of fecal neopterin in patients with Crohn's disease or ulcerative colitis was shown to be a biomarker for disease activity. As shown, measurement of fecal neopterin may be utilized in conjunction with measurement of biomarkers including fecal Calprotectin and lactoferrin, as well as blood measures including erythrocyte sedimentation rate (ESR) and C-reactive protein (CRP).

### SUMMARY

Disclosed are methods for determining disease activity in a patient having or at risk for developing inflammatory bowel disease (IBD). The methods typically include measuring neopterin concentration in a fecal sample from the patient.

In some embodiments, the patient has or is at risk for developing Crohn's disease, which may include active or inactive Crohn's disease. In further embodiments, the patient may have isolated ileal disease, isolated ileocolonic disease, or isolated colonic disease.

In some embodiments, the patient has or is at risk for developing ulcerative colitis, which may include active or inactive ulcerative colitis. In further embodiments, the patient may have pancolitis or left-sided colitis.

The methods typically include measuring neopterin concentration in a fecal sample. In some embodiments, the methods may include measuring a neopterin concentration of at least about 2 ng neopterin/g fecal sample (or at least about 5 ng neopterin/g fecal sample, or at least about 10 ng neopterin/g fecal sample, or at least about 20 ng neopterin/g fecal sample, or at least about 30 ng neopterin/g fecal sample, or at least about 40 ng neopterin/g fecal sample, or at least about 50 ng neopterin/g fecal sample, or at least about 60 ng neopterin/g fecal sample, or at least about 70 ng neopterin/g fecal sample, or at least about 80 ng neopterin/g fecal sample, or at least about 90 ng neopterin/g fecal sample, or at least about 100) ng neopterin/g fecal sample, or at least about 110 ng neopterin/g fecal sample, or at least about 120 ng neopterin/g fecal sample).

The disclosed methods preferably exhibit a relatively high selectivity and sensitivity with respect to determining whether the patient has or is at risk for developing an IBD as disclosed herein. In some embodiments, the methods exhibit a selectivity and/or sensitivity of at least about 80%, 85%, 90%, or 95%.

In some embodiments, neopterin concentration may be measured via contacting a fecal sample with an anti-neopterin antibody. The methods may include measuring neopterin concentration via performing an enzyme linked immunosorbent assay.

The methods may include measuring other factors associated with IBD. In some embodiments, the methods further include measuring hemoglobin concentration in a whole blood sample from the patient. In further embodiments, the methods include performing a hematocrit on a whole blood sample from the patient.

The method may include measuring concentration of factors in the fecal sample in addition to neopterin. In some embodiments, the methods include measuring lactoferrin concentration in the fecal sample.

In some embodiments, the methods further may include measuring factors present in a serum sample from a patient. For example, the methods may include measuring albumin concentration in a serum sample from the patient. In further embodiments, the methods may include measuring erythrocyte sedimentation rate in a serum sample from the patient. In even further embodiments, the methods may include measuring C reactive protein concentration in a serum sample from the patient.

### BRIEF DESCRIPTION OF THE DRAWINGS

FIG. 1. AUC Plot Showing Specificity and Sensitivity for Fecal and Serum Neopterin Concentrations to Predict Clinically Active Ulcerative Colitis (SCCAI>5).

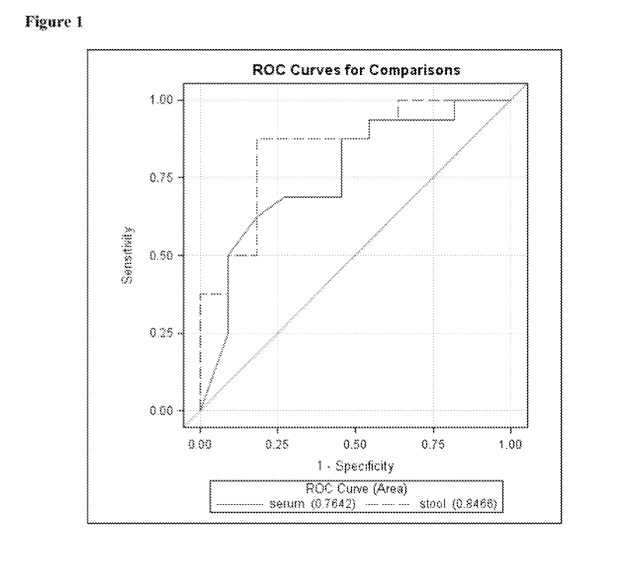

### DETAILED DESCRIPTION

The present invention is described herein using several definitions, as set forth below and throughout the application.

Unless otherwise specified or indicated by context, the terms “a”, “an”, and “the” mean “one or more.” For example, “an aromatase inhibitor” should be interpreted to mean “one or more aromatase inhibitors.”

As used herein, “about,” “approximately,” “substantially,” and “significantly” will be understood by persons of ordinary skill in the art and will vary to some extent on the context in which they are used. If there are uses of these terms which are not clear to persons of ordinary skill in the art given the context in which they are used, “about” and “approximately” will mean plus or minus ≦10% of the particular term and “substantially” and “significantly” will mean plus or minus >10% of the particular term.

As used herein, the terms “include” and “including” have the same meaning as the terms “comprise” and “comprising.” For example, “a method that includes a step” should be interpreted to mean “a method that comprises a step.”

As used herein, a “patient” may be interchangeable with “subject” or “individual” and means an animal, which may be a human animal, in need of treatment.

A “patient in need thereof” may include a patient having or at risk for developing an inflammatory bowel disease. For example, a “patient in need thereof” may include a patient having or at risk for developing Crohn's disease, which may include active or inactive Crohn's disease, and which may include isolated ileal disease, isolated ileocolonic disease, or isolated colonic disease. A “patient in need thereof” may include a patient having or at risk for developing ulcerative colitis, which may include active or inactive ulcerative colitis, and which may include pancolitis or left-sided colitis

As used herein, “neopterin” refers to a pyrazino-[2,3-d]pyrimidine compound having a formula:

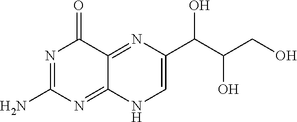 [SMILES: Nc1nc2[nH]cc(C(O)C(O)CO)nc-2c(=O)n1]

Methods for detecting neopterin in patient samples are known in the art. (See. e.g., U.S. Pat. Nos. 7,435,384; 7,135,295; 6,975,402; 6,376,195; 6,258,551; 6,013,457; 5,874,216; 5,733,437; 5,730,857; and U.S. Published Application Nos. 2011-0053163; 2010-0311068; 2010-0112599; 2009-0104602; the contents of which are incorporated herein by reference in their entireties). Methods for detecting neopterin may include, but are not limited to enzyme-linked immunosorbent assays (ELISA). (See “Soluble products of immune activation: Neopterin” Fuchs D, et al. Institute of Medical Chemistry and Biochemistry, University of Innsbruck, Innsbruck, Austria (In: Manual of Clinical Laboratory Immunology. 4th Edition (Rose R R, deMacario E C, Fahey J L, Friedman H, Penn G M, editors), The American Society for Microbiology, Washington D.C., 1992, 251-255); “Commercial enzyme-linked immunosorbent assay for neopterin detection in blood donations compared with RIA and HPLC.” Mayersbach P, et al. Central Inst. Blood Transfusion and Immunology, University Hospital, Innsbruck, Austria (Clin Chem 1994; 40: 265-266); and “Evaluation of a new simple and rapid enzyme-linked immunosorbent assay kit for neopterin determination.” Westermann J, et al. Immuno-Biological Laboratories GmbH, Hamburg, German (Clin Chem Lab Med 2000; 38: 345-353; the contents of which are incorporated herein by reference in their entireties.) Kits for detecting neopterin via ELISA are available commercially.

The methods disclosed herein may be performed using a patient sample which may include a fecal sample (or stool sample) or a blood sample, which may include a whole blood sample or a blood product such as plasma or serum. Methods and devices for collecting fecal samples and for detecting analytes in fecal samples are known in the art. (See, e.g., U.S. Pat. Nos. 7,833,794; 7,781,170; 7,780,915; 7,772,012; 7,736,660; 7,449,340; 7,338,634; 7,288,413; 7,252,955; 6,872,540; 6,727,073; 6,703,206; 6,640,355; 6,531,319; 6,063,038; 6,057,166; 5,730,147; 5,344,762; 5,331,973; 5,250,418; 5,198,365; 5,190,881; 5,171,528; 5,094,956; 5,066,463; 5,064,766; 4,920,045; 4,789,629; 4,645,743; and 4,309,782; the contents of which are incorporated herein by reference in their entireties).

The methods disclosed herein preferably exhibit a relativity high sensitivity and specificity. As used herein, “sensitivity” refers to the proportion of actual positives which are correctly identified as such (e.g., the percentage of sick people who are correctly identified as having the condition). As used herein, “specificity” refers to the proportion of negatives which are correctly identified (e.g., the percentage of healthy people who are correctly identified as not having the condition). Sensitivity may be defined by the equation “number of true positives”/(“number of true positives”+“number of false negatives”), and specificity may be defined by the equation “number of true negatives”/(“number of true negatives”+“number of false positives”), where “true positive” means a sick person correctly diagnosed as sick; “false positive” means a healthy person incorrectly identified as sick; “true negative” means a healthy person correctly identified as healthy; and “false negative” means a sick person incorrectly identified as healthy.

### Example

The following Example is illustrative and is not intended to limit the scope of the claimed subject matter.

Neopterin Concentration as an Index of Disease Activity in Crohn's Disease and Ulcerative Colitis

Background

Clinical indices for disease activity in inflammatory bowel disease (IBD) have received substantial and well-founded criticism for their well-documented subjectivity and non-specificity, in that scores may be affected by a myriad of issued not directly related to IBD.^{1,2} Various biomarkers have been proposed to objectively evaluate disease activity, including ESR, CRP, and, more recently, fecal calprotectin and lactoferrin. However, sensitivity has been a concern for each.^{3,4} Although numerous reports have described the ability of individual biomarkers in serum or feces to predict or confirm clinical disease activity,⁵⁻⁸ Jones and colleagues found no correlations between the CDAI and endoscopic mucosal appearance, or between the CDAI and serum CRP, fecal lactoferrin, or calprotectin.⁹ A combination of biomarkers may be the most useful for prediction or confirmation of clinical disease activity and endoscopically-visible inflammation.²

Neopterin, a pyrazino-[2,3-d]-pyrimidine compound, is a metabolite of cyclic guanosine monophosphate^{10,11} that is released by activated T-lymphocytes and macrophages following induction by γ-interferon.¹²⁻¹⁴ Homocysteine stimulates macrophages to release neopterin in vitro.¹⁵ Previous studies have reported separately that urine and serum neopterin concentrations are increased in patients with active Crohn's disease^{10,16-19} or active ulcerative colitis.²⁰ Urine neopterin concentration was used to appropriately stratify patients with active moderately severe Crohn's disease, active severe disease, mild disease, and quiescent disease, although the study used a single physician's subjective assessment as the standard of comparison.¹⁰ In another study, serum TNF-α concentration correlated with whole-blood neopterin concentration in a univariate analysis (r=0.73, p<0.0001).¹⁹ In patients with ulcerative colitis, a small study reported highly significant correlations were observed between serum neopterin concentration and both ESR and the number of daily bowel movements, weaker, although still statistically significant correlations, were observed with increased body temperature and presence or absence of anemia.²⁰ Elevated urine neopterin concentrations normalized when clinical remission was achieved. Increased fecal neopterin concentration has been observed in association with inflammation and increased intestinal permeability in children in Gambia infected with Giardia lamblila.²¹ Fecal neopterin concentration has not previously been evaluated in IBD. Accordingly, we evaluated fecal, serum, and urine neopterin concentration as an independent biomarker of clinical disease activity in patients with Crohn's disease or ulcerative colitis.

Methods

Subjects: We prospectively studied 70 outpatients (51% male mean age 39.2±14.0 years) with Crohn's disease (33 clinically in remission, 37 active) and 52 outpatients (58% male, mean age 39.8±12.2 years) with ulcerative colitis (29 clinically in remission, 23 active). For patients with Crohn's disease, disease distribution was as follows: 42% isolated ileal, 20% ileocolonic, 38% isolated colonic. For patients with ulcerative colitis, disease distribution was as follows: 74% pancolitis and 24% left-sided colitis. Patients with disease limited to proctitis were not included. Neopterin concentration was analyzed in feces, serum, and urine samples. Simultaneously, Hgb and Hct were measured in whole blood samples, fecal samples were analyzed for lactoferrin, and serum samples were also analyzed for albumin, C-reactive protein (CRP), and erythrocyte sedimentation rate (ESR). Clinical indices were calculated when patients were enrolled. Crohn's disease was considered active if the Harvey Bradshaw Index (HBI) was ≧5;²² ulcerative colitis was considered active if the Simple Clinical Colitis Activity Index (SCCAI) was >3.²³

Healthy individuals from the Orange County area of Southern California were recruited as control subjects for neopterin measurements. Individuals with a known history of or first-degree relative with inflammatory bowel disease or any autoimmune disease were excluded, although volunteers for serum samples were not queried for this history. The control subjects included 21 men and 30 women aged 18-65 years (median 36 years) for fecal neopterin, 56 men and 60 women aged 18-61 years (median 37 years) for serum neopterin, and 60 men and 60 women aged 18-65 years (median 36 years) for urine neopterin.

Laboratory Testing: 20 ml of stool, blood, and urine were collected from each subject at an outpatient clinic visit. In some instances, when the subject was unable to provide a fecal sample, feces were collected the following day. Blood was centrifuged at 2000×g for 10 minutes, and the serum obtained was frozen at −72° C. prior to analysis. 20 ml urine and weighed fecal samples were frozen as well. Serum and urine neopterin concentration was measured using an enzyme-linked immunosorbent assay (Neopterin ELISA kit IB29125; Immuno-Biological Laboratories. Minneapolis, Minn.). After defrosting but just prior to analysis, 0.5 ml of 0.9% normal saline was added to the fecal sample, which was agitated for 30 min and then centrifuged at 3500×g for 20 min. The supematant was collected and the neopterin concentration was by ELISA as noted previously. Neopterin concentration was expressed per g of dried stool.²¹ ESR (mm/hr), Hgb, Hct, CRP, and serum alb concentrations were determined in blood samples using standard techniques.

The ELISA-based assay (IBD-Check, Techlab, Blacksburg, Va.) was used to detect elevated levels of fecal lactoferrin. Briefly, 96-well plates coated with polyclonal antibody against lactoferrin were used to bind lactoferrin present in fecal specimen. Bound lactoferrin was detected with specific polyclonal antibodies conjugated to horseradish peroxidase. Following the addition of substrate, color was detected due to the enzyme-antibody-antigen complexes that form in the presence of lactoferrin. The optical density (OD) was measured at 450 with reference at 630 nm. An OD value ≧0.300 indicated elevated (abnormal) level of lactoferrin.

For Crohn's disease, the following disease activity scores were determined: the Capetown Index²⁴ and the Disease Activity Index (DAI) for ulcerative colitis,²⁵ was adapted for use in Crohn's disease. The Crohn's Disease Activity Index (CDAI)^{22,23} was not calculated because subjects were enrolled at the time of specimen collection at routine clinic visits and a “retrospective” CDAI has not been validated.

For ulcerative colitis, the following disease activity scores were determined: the DAI,²⁵ and colonoscopy, where the endoscopic disease severity was rated on a scale of 0-3 with Grade 1 absent vascular pattern, mild friability, and fine granularity; Grade 2 marked friability and erosions; and Grade 3 marked friability, coarse granularity, erosions, spontaneous bleeding and ulcerations. The endoscopic extent of disease was recorded. A new scoring system (BIDS) that includes subjective and objective clinical components, biomarkers, and other laboratory testing was utilized for both Crohn's disease and ulcerative colitis (Table 1).

Statistical Analysis

Due to non-normality of data, medians and percentiles are presented. Mann-Whitney tests, Kruskal-Wallis tests, and Spearman's correlation coefficients were employed. Spearman's rank correlations (rₛ) were estimated between serum, urine, and fecal neopterin concentrations for CRP, ESR, CDAI, and Capetown Index for Crohn's disease, and CRP, ESR, DAI, and endoscopic mucosal appearance for ulcerative colitis.

Results

Fecal, urine, and serum neopterin concentrations for patients with Crohn's disease or ulcerative colitis, as well as for healthy controls are presented in Table 2. Fecal neopterin concentrations were higher in patients with active or inactive Crohn's disease than in control subjects (Table 2). However, they did not statistically correlate with any other the serum or fecal biomarker, or clinical index of disease activity. Fecal neopterin concentration was higher in patients with active Crohn's colitis, although this trend did not reach statistical significance. The median fecal neopterin concentrations for ileal, ileocolonic, and colonic disease were 87.6, 54.5, and 109.7 ng/g, respectively.

There was a nonsignificant trend towards increased serum neopterin in patients with active Crohn's disease relative to those whose disease was in remission. However, the control group exhibited median serum neopterin concentrations significantly greater than those of patients with either clinically active or inactive Crohn's disease. Serum neopterin concentrations were negatively correlated with albumin (rₛ=−0.38, p<0.01) and positively correlated with CRP (rₛ=0.43, p<0.01), and ESR (rₛ=0.32, p=0.02), but were not correlated with the Capetown index, Hgb, Hctt, or fecal lactoferrin concentration.

Urine neopterin concentration in patients with either clinically active or inactive disease did not differ from that of controls, (Table 2). However, it was weakly, although significantly negatively correlated with Hgb (rₛ=−0.37, p<0.01), Hct (rₛ=−0.34, p=0.01), albumin (rₛ=−0.36, p=0.01), and positively correlated with the Capetown index (rₛ=0.27, p=0.04), CRP (rₛ=0.38, p<0.01), and ESR (rₛ=0.31, p=0.02).

Hgb, Hct, albbumin, CRP, ESR, and lactoferrin were not useful in isolation to confirm the presence of clinically active Crohn's disease in our outpatient population, although the clinical indices of disease activity (BIDS, CD-DAI, and the Capetown Index) were all important indicators of the presence of clinically active disease as suggested by the HBI. (Table 3).

Among patients with ulcerative colitis, fecal neopterin concentration was significantly greater in those with clinically active disease than in those with clinically inactive disease and control subjects (Table 2). A fecal neopterin concentration cutoff value of 98.4 ng/g stool provided a sensitivity of 87.5% for the prediction of active ulcerative colitis at a specificity of 81.8% (FIGURE). A non-significant trend was observed towards greater fecal neopterin concentration in patients with clinically active pancolitis when compared with those whose disease was active only in the left colon: median fecal neopterin concentration for left-sided colitis and pancolitis were 59.9 and 135.2 ng/g, respectively. Fecal neopterin was positively correlated with the ESR (rₛ=0.40, p=0.04), but not with the DAI, Hgb, Hct, alb, or CRP.

Serum neopterin concentration was also significantly greater in patients with clinically active ulcerative colitis than in those whose disease was in clinical remission, but still lower than controls (Table 2). Serum neopterin concentration was negatively correlated with Hct (rₛ=−0.30, p=0.04) and alb (rₛ=−0.43, p<0.01), while positively correlated with the CRP (rₛ=0.37, p=0.02), and ESR (rₛ=0.35, p=0.02). No significant correlation was detected with Hgb or the DAI.

We did not detect any difference in urine neopterin concentrations between either patients with clinically active or clinically inactive ulcerative colitis, or those with clinically active disease compared to controls. Urine neopterin concentration was weakly but significantly negatively correlated with Hgb (rₛ=−0.37, p 0.02), Hct (rₛ=−0.43, p=0.01), and albumin concentration (rₛ=−0.38, p=0.01), but not with the DAI, CRP, ESR, or fecal lactoferrin level.

Hgb, Hct, albumin, CRP, and ESR by themselves, were not useful for confirming the presence of clinically active ulcerative colitis in our outpatient population, although an elevated fecal lactoferrin and the clinical indices of disease activity (BIDS and the DAI) were all important indicators of the presence of clinically active disease as suggested by the SCCAI (Table 3).

Urine, serum, and fecal neopterin, SSCAI, CRP, and ESR all failed to correlate with endoscopic scores. Serum Hgb and alb were negatively correlated with endoscopic score (rₛ=−0.36, p=0.055; and rₛ=−0.37, p=0.05, respectively).

Discussion

Prior to our study, fecal neopterin concentration had not been evaluated in patients with inflammatory bowel disease. We found it to be significantly increased in patients with either active or inactive Crohn's disease as well as those with active ulcerative colitis compared with control subjects. Although fecal neopterin concentration could reliably distinguish between clinically active and inactive ulcerative colitis, it did not distinguish between clinically active and inactive Crohn's disease. The nonsignificant trend we observed for higher fecal neopterin concentrations in patients with ulcerative colitis whose disease was judged to be in clinical remission, as well as the clear elevation in patients with Crohn's disease who were in clinical remission, suggests the likelihood of ongoing subclinical intestinal and/or colonic inflammation that was not evident clinically, or by changes in other biomarkers. Our data also suggest that fecal neopterin concentration is greater in patients with active disease that is limited to the colon in Crohn's colitis and ulcerative colitis and also increases as more of the colon is affected in patients with ulcerative colitis. However, a larger study would be needed to confirm those observations.

Our data cannot confirm previous reports of the utility of serum neopterin measurement in differentiating patients with IBD from normal individuals. The inexplicably elevated concentrations found in our control subjects were compatible with concentrations anticipated in active IBD. We could not exclude the possibility that several of our control subjects had asymptomatic autoimmune disease, viral illness, or even IBD. Therefore, additional investigation of the use of serum neopterin concentration as a biomarker for disease activity in both Crohn's disease and ulcerative colitis will need to be undertaken.

Urine neopterin concentration failed to distinguish either clinically active or inactive patients from healthy controls. It is unclear why our data were inconsistent with previous studies that reported elevated urine neopterin concentrations in patients with either active Crohn's disease or active ulcerative colitis.^{16,18,20} Our data would question the sensitivity of urine neopterin concentration in the detection of active IBD.

Although differences were seen in distributions of serum neopterin concentration between both Crohn's disease and ulcerative colitis groups and controls, it is unclear if there is a single mechanism that might explain this observation. Patients with active Crohn's disease in our study exhibited serum neopterin concentrations equal to or somewhat greater than in two previous French studies, but our control population had substantially greater concentrations than reported in these studies.^{19,26} It is not clear why this occurred. Our data are the first reported of which we are aware that describe serum neopterin concentrations in patients with ulcerative colitis. Additional data is required to ascertain normal serum neopterin concentration in given populations as well as to determine factors other than inflammation that may affect it.

In contrast to previous studies, an elevated fecal lactoferrin was not predictive of active Crohn's disease, although it was for ulcerative colitis. It has been previously suggested that fecal lactoferrin correlates better with ulcerative colitis activity.²⁷ In our study, elevated CRP was not predictive of activity for either disease, although there was a trend towards an elevated ESR being a marker of disease activity. The new BIDS had the greatest correlation with disease activity for both CD and UC when the HBI was used as the gold standard, although the Capetown index and DAI (when applied to patients with Crohn's disease as well as more conventionally in patients with UC) were also predictive of disease activity.

Because fecal neopterin concentration was not associated with other serum biomarkers or any clinical indices of disease activity, it may be a promising independent biomarker for Crohn's disease and suggests a need for further investigation. None of the biomarkers studied (CRP, ESR, fecal lactoferrin, Hgb, Hct) were useful in the prediction or confirmation of active Crohn's disease, although all three of the clinical scoring indices were. In patients vith active ulcerative colitis, only fecal lactoferrin, in addition to neopterin was a marker for disease activity although as in the case with Crohn's disease, clinical disease activity indices did predict and confirm the presence of active disease. Further investigation will be needed to determine whether elevated fecal neopterin concentration in an otherwise asymptomatic individual is predictive of relapse. Furthermore, the potential role for fecal, and possibly serum neopterin concentration in monitoring response to treatment or for determining treatment failure will need to be delineated.

Summary

Various biomarkers have been proposed to objectively evaluate disease activity, including ESR. CRP, and, more recently, fecal calprotectin and lactoferrin. However, sensitivity has been a concern for each. Neopterin, a pyrazino-[2,3-d]-pyrimidine compound, is a metabolite of cyclic guanosine monophosphate that is released by activated T-lymphocytes and macrophages following induction by γ-interferon. Urine or serum neopterin concentration may be useful in predicting or confirming the presence of active Crohn's disease or ulcerative colitis. Increased fecal neopterin concentration has been observed in association with inflammation and increased intestinal permeability in children in Gambia infected with Giardia lamblia.

As shown here, fecal neopterin concentration is significantly increased in patients with either clinically active or inactive Crohn's disease. Fecal neopterin is significantly increased in patients with active ulcerative colitis and reliably differentiates between patients with active and inactive disease. The degree of elevation in fecal neopterin concentration may be in part related to location and extent of disease. Significant elevation of fecal neopterin in patients with clinically inactive Crohn's disease suggests the presence of on-going inflammation.

### ABBREVIATIONS

Hgb (hemoglobin), Hct (hematocrit), Alb (albumin), CRP (C reactive protein), ESR (erythrocyte sedimentation rate), CD (Crohn's disease), UC (ulcerative colitis), CDAI (Crohn's disease activity index), IBD (inflammatory bowel disease), BIDS (Buchman Inflammatory Bowel Disease Index), DAI (Disease Activity Index), SCCAI (Simple Clinical Colitis Activity Index)

### REFERENCES

1. Jorgensen L G, Fredholm L. Hyloft Peterson P, et al. How accurate are clinical activity indices for scoring of disease activity in inflammatory bowel disease (IBD)? Clin Chem Lab Med 43:403-11, 2005.
2. Langhorst J, Elsenbrunch S, Koelzer S, et al. Noninvasive markers of assessment of intestinal inflammation in inflammatory bowel disease: performance of fecal lactoferrin, calprotectin, and PMN-elastase, CRP, and clinical indices. Am J Gastroenterol 103:162-9, 2008.
3. Florin T H, Paterson E W, Fowler E V, et al. Clinically active Crohn's disease in the presence of a low C-reactive protein. Scan J Gastroenterol 41:306-11, 2006.
4. Denis M A, Reenaers C, Fontaine F, et al. Assessment of endoscopic activity index and biological inflammatory markers in clinically active Crohn's disease with normal C-reactive protein level. Inflamm Bowel Dis 13:1100-05, 2007.
5. Desai D, Faubion W A, Sandborne W J. Review article: biological activity markers in inflammatory bowel disease. Aliment Pharmacol Ther 25:247-55, 2007.
6. Sutherland A D, Gearry R B, Frizelle F A. Review of fecal biomarkers in inflammatory bowel disease. Dis Colon Rectum 51:1283-91, 2008.
7. Moscandrew M E, Loftus E V Jr. Diagnostic advances in inflammatory bowel disease (imaging and laboratory). Curr Gastroenterol Rep 11:488-95, 2009.
8. Beniwal P, Harrell L. The status of diagnostic markers for inflammatory bowel disease. Curr Gastroenterol Rep 12:479-84, 2010.
9. Jones J, Loftus E V, Panaccione R, et al. Relationships between disease activity and serum and fecal biomarkers in patients with Crohn's disease. Clin Gastroenterol Hepatol 6:1218-24, 2008.
10. Judmaier G, Meyersbach P, Weiss G, et al. The role of neopterin in assessing disease activity in Crohn's disease: classification and regression trees. Am J Gastroenterol 88:706-11, 1993.
11. Andert S E, Muller M M. Neopterin: biochemistry and clinical usefulness. J Int Fed Clin Chem 7:70-76, 1995.
12. Seki T, Sugie N, Joh K, et al. The regulation of neopterin production by cytokines. Pteridines 7:5-9, 1996.
13. Huber C, Batchelor J R, Fuchs D, et al. Immune response associated production of neopterin. Release from macrophages primarily under control of interferon-gamma. J Exp Med 160:310-316, 1983.
14. Huber C. Fuchs D, Hausen A, et al. Pleridines as a new marker to detect human T cells activated by allogeneic or modified self major histocompatability complex (MHC) determinants. J Immunol 130:1047-1050, 1983.
15. Schroecksnadel K, Frick B, Wirleitner B, et al. Homocysteine accumulates in supernatents of stimulated human peripheral blood mononuclear cells. Clin Exp Immunol 134:53-56, 2003.
16. Prior C H, Bollbach R, Fuchs D. et al. Urinary neopterin, a marker of clinical activity in patients with Crohn's disease. Clin Chim Acta 155:11-22, 1986.
17. Konecny M, Ehrmann J. Bartek J, et al. The level of neopterin in blood serum as one of the possible prognostic markers of Crohn's disease activity. Acta Univ Palacki Olomuc Fac Med 140:47-48, 1996.
18. Nancey S, Perret-Liaudet A, Moussata D, et al. Urinary neopterin is a valuable tool in monitoring Crohn's disease activity. Inflamm Bowel Dis 14:1548-54, 2008.
19. Reimund J M, Arondel Y. Escalin G, et al. Immune activation and nutritional status in adult Crohn's disease patients. Digestive Liver Dis 37:424-431, 2005.
20. Niederwieser D, Fuchs D, Hausen A, et al. Neopterin as a new biochemical marker in the clinical assessment of ulcerative colitis. Immunobiol 170:320-326, 1985.
21. Campbell D I, McPhail G, Lunn P G, et al. Intestinal inflammation measured by fecal neopterin in Gambian children with enteropathy: association with growth failure, Giardia lamblia, and intestinal permeability. J Pediatr Gastroenterol Nutr 39:153-157, 2004.
22. Harvey R F, Bradshaw J M. A simple index of Crohn's disease activity. Lancet 1:514, 1980.
23. Walmsley, R S, Ayres R C S, et al. A simple clinical colitis activity index. Gut 43:29-32, 1998.
24. Wright J P, Marks I N, Parfitt A. A simple index of Crohn's disease activity. The Cape Town index. South African Med J 68:502-503, 1985.
25. Sutherland L R, Martin F, Greer S, et al. 5-aminosalicylic acid enema in the treatment of distal ulcerative colitis, protosigmoiditis, and proctitis. Gastroenterology 92:1894-1898, 1987.
26. Duclos B, Reimund J M, Lang J M, et al. Activation des cellules mononucleees dans la maladie de Crohn. Gastroenterol Clin Biol 14:22-7, 1990.
27. Gisbert J P, McNicholl A G, Gomollon F. Questions and answers on the role of fecal lactoferrin as a biological marker in inflammatory bowel disease. Inflamm Bowel Dis 15:1746-54, 2009.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

TABLE 1
BIDS System

| | Frequency/ | |
|---|---|---|
| | Severity | Score |
| Number of Daily Bowel | <3 | 0 |
| Movements | 3-5 | l |
| | 6-10 | 2 |
| | 11-15 | 3 |
| | <15 | 4 |
| Abdominal Pain | 0-4 rating scale | 0-4 |
| Fatigue | 0-4 rating scale | 0-4 |
| Nonintentional Weight Loss | <1 | 0 |
| in Last 3 months (lbs) | 1-5 | 2 |
| | 6-10 | 3 |
| | >10 | 4 |
| Extra-Intestinal | peripheral arthralgias | 5 points |
| Manifestations of IBD | uveitis/episcleritis | each |
| | erythema nodosum/pyoderma | |
| | one of more fistulas of any | |
| | type | |
| Fever | >100.° F. | 3 |
| Abdominal Mass on Physical | | 3 |
| Examination | | |
| Serum Albumin | >3.5 | 0 |
| Concentration (g/L) | 3.0-3.4 | 1 |
| | 2.5-2.9 | 2 |
| | 2.0-2.4 | 3 |
| | <2.0 | 4 |
| Leukocytosis (per ml³) | <12,000 | 0 |
| | 12,000-15,000 | 1 |
| | 15,001-20,000 | 3 |
| | >20,000 | 4 |
| Hemoglobin (g/dl) | >12 | 0 |
| | 10-12 | 1 |
| | 8-9.9 | 2 |
| | <8 | 3 |
| ESR (mm/hr) | <18 | 0 |
| | 18-24 | 1 |
| | 25-35 | 2 |
| | 36-50 | 3 |
| | >50 | 4 |
| CRP (mg/dl) | <0.9 | 0 |
| | 0.9-1.2 | 1 |
| | 1.3-2.5 | 2 |

TABLE 2
Median, 25^{th} and 75^{th} Percentiles, Minimum, and Maximum Fecal, Serum,
and Urine Neopterin Concentrations in Controls and Patients with
Active and Inactive Inflammatory Bowel Disease.

| | | | Crohn's | Crohn's | UC | UC |
|---|---|---|---|---|---|---|
| | | Controls^{a,b,c} | Active^{d,e,f} | Inactive^{g,h,i} | Active^{j,k,l} | Inactive^{m,n,o} |
| Fecal | Median | 12.0 | 87.2 | 96 | 135.2 | 62.7 |
| Neopterin | 25^{th} | 6.7 | 66.3 | 48.1, | 102.5, | 46.7 |
| (ng/g stool) | 75^{th} | 42.3 | 150.9)† | 139.3)† | 235.9†‡ | 96.3† |
| | Min | 3.4 | 20.3 | 20.1 | 55.6 | 7.3 |
| | Max | 379.3 | 322 | 262.4 | 248 | 182 |
| Serum | Median | 4.56 | 3.35 | 2.85 | 3.8 | 2.3 |
| Neopterin | 25^{th} | 3.44 | 2.75 | 1.95 | 2.6 | 2.0 |
| (nmol/L) | 75^{th} | 5.61 | 5.6 | 3.85† | 9.5‡ | 3.1† |
| | Min | 2.29 | 2.0 | 1.5 | 1.5 | 1.5 |
| | Max | 14.60 | 19 | 12.4 | 21.3 | 57.3 |
| Urine | Median | 94.0 | 98.5 | 93 | 90 | 108 |
| Neopterin | 25^{th} | 69 | 75 | 58 | 55.5 | 74 |
| (μmol/mol | 75^{th} | 128 | 199 | 132 | 217 | 224 |
| creat) | Min | 15 | 57 | 37 | 40 | 42 |
| | Max | 2173 | 558 | 956 | 614 | 1061 |

†Wilcoxon rank-sum test compared to Controls significant at Bonferroni-corrected p < 0.05.
‡Wilcoxon rank-sum test compared to Inactive Disease significant at Bonferroni-corrected
p < 0.05
^{a}n = 21 M, 28 F aged 18-63 years (fecal)
^{b}n = 60 M, 55 F aged 18-65 years (urine)
^{c}n = 60 M, 60 F aged 18-61 years (serum)
^{d}n = 25 (fecal)
^{e}n = 30 (urine)
^{f}n = 28 (serum)
^{g}n = 18 (fecal)
^{h}n = 29 (urine)
ⁱn = 28 (serum)
^{j}n = 11 (fecal)
^{k}n = 20 (urine)
^{l}n = 22 (serum)
^{m}n = 16 (fecal)
ⁿn = 24 (urine)
^{o}n = 26 (serum)

TABLE 3
Median and Range of Laboratory Values and Clinical Activity Indice
Scores for Patients with Clinically Active or Inactive IBD

| | | | | | p- |
|---|---|---|---|---|---|
| | Remission | | Active | | value |
| CD Patients | | | | | |
| Hgb (g/dl) | 13.7 | [10.0, 16.6] | 13.5 | [10.2, 16.2] | 0.522 |
| Hct (%) | 39.7 | [28.5, 47.4] | 39.0 | [31.7, 47.3] | 0.986 |
| Alb (g/L) | 3.8 | [2.5, 4.7] | 3.8 | [2.6, 35.0] | 0.478 |
| CRP (mg/dl) | 0.6 | [0.5, 7.8] | 1.0 | [0.5, 4.8] | 0.170 |
| ESR (mm/hr) | 10 | [0, 76] | 18 | [0, 83] | 0.079 |
| Lactoferrin | 4/13 | (31%) | 8/14 | (57%) | 0.168 |
| (OD > 0.3) | | | | | |
| BIDS | 3 | [0, 18] | 9 | [2, 20] | <0.001 |
| CD- DAI | 49 | [0, 248] | 122 | [8, 371] | <0.001 |
| Capetown Index | 3 | [0, 10] | 7 | [2, 44] | <0.001 |
| UC patients | | | | | |
| Hgb (g/dl) | 14.0 | [9.0, 16.1] | 13.8 | [7.6, 15.7] | 0.598 |
| Hct (%) | 40.3 | [27.4, 52.1] | 40.8 | [0.5, 46.7] | 0.690 |
| Alb (g/L) | 4.0 | [2.8, 4.4] | 3.8 | [1.8, 4.9] | 0.104 |
| CRP (mg/dl) | 0.5 | [0.5, 8.5] | 0.6 | [0.5, 9.0] | 0.643 |
| ESR (mm/hr) | 5 | [0, 44] | 18 | [0, 65] | 0.079 |
| Lactoferrin | 4/9 | (44%) | 8/8 | (100%) | 0.029 |
| (OD > 0.3) | | | | | |
| BIDS | 2 | [0, 9] | 11 | [1, 22] | 0.001 |
| UC- DAI | 5 | [2, 9] | 7 | [4, 9] | 0.005 |

1.-20. (canceled)

21. A kit comprising:
(a) components for collecting and preparing a fecal sample from the patient;
(b) components for measuring neopterin concentration in the fecal sample; and
(c) components for measuring calprotectin or lactoferrin in the fecal sample.

22. The kit of claim 21, wherein the components for measuring neopterin concentration in the fecal sample comprise an anti-neopterin antibody.

23. The kit of claim 21, wherein the components for measuring neopterin concentration in the fecal sample comprise an enzyme-linked immunosorbent assay.

24. The kit of claim 21, wherein the components for measuring calprotectin or lactoferrin in the fecal sample comprise an anti-calprotectin antibody or an anti-lactoferrin antibody.

25. The kit of claim 21, wherein the components for measuring calprotectin or lactoferrin in the fecal sample comprise an enzyme-linked immunosorbent assay.

26. The kit of claim 21, wherein the kit further comprises:
(d) components for collecting and preparing a whole blood sample from the patient; and
(e) components for measuring hemoglobin concentration in the whole blood sample or components for performing a hematocrit on the whole blood sample.

27. The kit of claim 21, wherein the kit further comprises:
(d) components for collecting and preparing a serum sample from the patient; and
(e) components for measuring albumin concentration or C reactive protein concentration in the serum sample.

28. The kit of claim 21, wherein the kit further comprises:
(d) components for collecting and preparing a serum sample from the patient; and
(e) components for measuring erythrocyte sedimentation rate in the serum sample.

29. A combination of kits comprising:
(a) a first kit for collecting and preparing a fecal sample from the patient;
(b) a second kit for measuring neopterin concentration in the fecal sample; and
(c) a third kit for measuring calprotectin or lactoferrin in the fecal sample.

30. The combination of claim 29, wherein the first kit for measuring neopterin concentration in the fecal sample comprises an anti-neopterin antibody.

31. The combination of claim 29, wherein the first kit for measuring neopterin concentration in the fecal sample comprises an enzyme-linked immunosorbent assay.

32. The combination of claim 29, wherein the second kit for measuring calprotectin or lactoferrin in the fecal sample comprises an anti-calprotectin antibody or an anti-lactoferrin antibody.

33. The combination of claim 29, wherein the second kit for measuring calprotectin or lactoferrin in the fecal sample comprises an enzyme-linked immunosorbent assay.

34. The combination of claim 29, wherein the combination further comprises:
(d) a fourth kit for collecting and preparing a whole blood sample from the patient; and
(e) a fifth kit for measuring hemoglobin concentration in the whole blood sample or components for performing a hematocrit on the whole blood sample.

35. The combination of claim 29, wherein the kit further comprises:
(d) a fourth kit for collecting and preparing a serum sample from the patient; and
(e) a fifth kit for measuring albumin concentration or C reactive protein concentration in the serum sample.

36. The combination of claim 29, wherein the kit further comprises:
(d) a fourth kit for collecting and preparing a serum sample from the patient; and
(e) a fifth kit for measuring erythrocyte sedimentation rate in the serum sample.
